(19) United States
(12) Patent Application Publication
(10) Pub. No.: US 2003/0114475 A1
(43) Pub. Date: Jun. 19, 2003

(54) Methods for the treatment of addiction

(75) Inventors: Barbara S. Fox, Wayland, MA (US); Kay Jorgenson D'Orlando, Wayland, MA (US)

Correspondence Address: HALE AND DORR, LLP, 60 STATE STREET, BOSTON, MA, 02109

(73) Assignee: Addiction Therapies, Inc., Wayland, MA

(21) Appl. No.: 10/285,038

(22) Filed: Oct. 31, 2002

Related U.S. Application Data
(60) Provisional application No. 60/334,706, filed on Oct. 31, 2001.

(51) Int. Cl. A61K 31/485; A61K 31/46

(52) U.S. Cl. 514/282; 514/304

(57) ABSTRACT

The present invention is directed to addiction treatment methods that include frequent or episodic dosing of medication coupled with a reinforcing behavior and/or stimulus. Performing a particular behavior and/or experiencing a particular stimulus in conjunction with administering medication causes patients to become engaged in therapy and focus on recovery.

### BACKGROUND OF THE INVENTION

1. Field of the Invention

The present invention is related to both the field of pharmacology and the field of behavior modification. More specifically, the invention relates to the treatment of drug addictions.

2. Summary of the Related Art

Addiction is a pressing individual and public health issue. Drug use has a severe negative impact on the mental and physical health of individual drug users. Drug use and addiction also present serious public health and safety hazards by playing a major role in violent crime and the transmission of infectious diseases, such as AIDS, hepatitis, and tuberculosis. Thus, treatment of addiction is an important goal in protecting the health and safety of individuals and society (see, Drug Abuse and Addiction Research, the Sixth Triennial Report to Congress from the Secretary of Health and Human Services, National Institute on Drug Abuse (1999)).

Addiction is a chronic, relapsing disease. It is believed that addiction is associated with extensive synaptic remodeling and that drug-taking behavior becomes associated with neural pathways in the dorsal striatum that control automatic, fixed tasks (see, Berke et al., Neuron 25:515-532 (2000)). Thus, for an addict, drug-taking behavior becomes difficult to control.

The majority of treatment methods for addiction are psychosocial. The goals of psychosocial treatment are to eliminate or reduce drug use during treatment and to decrease the likelihood of relapse after treatment has ended. These goals are accomplished by weakening the dependency on the addictive drug and by establishing competing dependencies on healthier behaviors. Some examples of psychosocial treatment methods are cognitive behavioral therapy, motivation to change, contingency management, individual psychotherapy, group therapy, in-patient programs, out-patient therapy, intensive out-patient therapy, extinction of conditioned craving, coping skills therapy, network therapy, aversion therapy, community reinforcement, and “twelve-step” programs. In some instances, such psychosocial therapies have been used to establish competing dependencies, or substitute behaviors (see, e.g., Vaillant, “Natural History of Addiction and Pathways to Recovery” in Principles of Addiction Medicine, Graham et al., eds., American Society of Addiction Medicine, 295-308 (1998)). Long-term success in preventing relapse sometimes depends on successfully establishing a competing dependency on an activity, such as exercise, or a group, such as Alcoholics Anonymous. Methods for overcoming undesired habits, including addictions, using a series of behavioral and pharmacological treatments also have been proposed (Eig, U.S. Pat. No. 6,333,357).

Approved pharmacological methods of treating addiction include slowly reducing doses of the addictive drug, making the addictive drug aversive or less reinforcing, and providing a replacement drug. For example, nicotine reduction therapy is employed using nicotine chewing gum, transdermal patches, nasal sprays, or inhalers. Alternative nicotine delivery devices such as toothpicks, lip balms, and lollipops also have been proposed. Replacement therapies, including bupropion hydrochloride, have also been employed for nicotine addiction. A combination therapy of naltrexone and nicotine for smoking cessation also has been proposed (U.S. Pat. No. 6,004,970). For drugs that, unlike nicotine, are subject to abuse and cause intoxication as well as inducing dependency (see Diagnostic and Statistical Manual of Mental Disorders, Fourth Edition, American Psychiatric Association (1994)), replacement therapies have been favored. Such therapies include treatment with methadone or levomethadyl acetate hydrochloride (LAAM) for addiction to heroin. Naltrexone has been used to block the effects of both heroin and alcohol, reducing their reinforcing activity. Disulfiram has been used to make alcohol aversive. Although a limited number of the aforementioned treatments, e.g., nicotine inhaler, include a behavioral component, most known pharmacological methods for treating addiction do not include a behavioral aspect.

Some existing pharmacological methods for treating addiction employ long-lasting depot formulations (e.g., transdermal nicotine patches or sustained release preparations of naltrexone) for medication delivery. In general, it is believed that a higher frequency of medication dosing corresponds to decreased patient compliance (e.g., Paes et al., Diabetes Care 20:1512-1517 (1997)). Thus, frequent dosing generally is not favored, and depot formulations are designed to improve patient compliance by decreasing the required dosing frequency (see, Claxton et al., Clin. Ther. 23:1296-1310 (2001)). Methods for increasing patient compliance by simplifying medication schedules or pairing medication dosing with high probability events, such as brushing teeth in the morning, also have been proposed (e.g., Thase et al., J. Clin. Psychiatry 62:32-41 (2001); Cramer et al., J. Nervous & Mental Disease 187:53-55(1999)). However, such methods and depot formulations separate pharmacological treatment from behavioral treatment by making medication delivery as invisible and easy as possible. This allows patients to be less aware of the existence of the addiction and the need to overcome it, rather than using medication delivery as a behavioral tool.

Despite recent advances, a continuing need exists for new and improved methods of treating addiction to help reduce the individual and public health problems associated with addictive disorders.

### SUMMARY OF THE INVENTION

The present invention addresses the foregoing problems by providing addiction treatment methods that include frequent or episodic dosing of medication coupled with a reinforcing behavior and/or stimulus. Such methods effectively address both the pharmacological and behavioral aspects of addiction, using reinforcing behaviors and stimuli associated with medicine delivery as tools to increase the efficacy of treatment. Performing a particular reinforcing behavior and/or experiencing a particular reinforcing stimulus, concurrently with administering medication, enhances patient engagement in treatment and supports patient mental and physical control over addiction. The reinforcing behavior or stimulus is a reminder of the existence of the addiction and the required process of working to overcome it. Further, the reinforcing behavior associated with medication delivery during treatment often creates a short-term alternate dependency or habit that facilitates extinction of the original addiction. Scheduled repetition of medication dosing imposes structure on the often chaotic lifestyle of a recovering addict, and provides an alternative activity to perform instead of behaviors associated with procuring and administering the addictive drug.

Accordingly, one aspect of the invention provides a method of treating a patient for addiction to a drug. The method includes providing a medication other than the drug for treating the addiction, identifying at least one reinforcing behavior, and instructing the patient to self-administer the medication and perform the reinforcing behavior concurrently between about three and about twenty times per day. “Concurrently” means that the reinforcing behavior is performed simultaneously with, or shortly before or after, self-administration of the medication. “Instructing” means indicating to a patient, for example, through oral directions from a physician or written directions accompanying a formulated product. A “drug” is a substance that is capable of causing a chemical or physical change in the body.

In some embodiments of the method, self-administration of the medication provides a reinforcing sensory stimulus. In certain embodiments, the sensory stimulus is unrelated to addiction-related self-administration of the drug. In particular embodiments, the sensory stimulus is unrelated to the pharmacological effect of the medication. In specific embodiments, the reinforcing behavior is included in self-administration of the medication.

In some embodiments, self-administration of the medication is more frequent than addiction-related self-administration of the drug. In certain embodiments, self-administration of the medication is performed at least five times per day, for example, at least eight times per day. In particular embodiments, the patient is instructed to self-administer the medication and perform the reinforcing behavior upon experiencing craving for the drug. The term “craving” refers to a strong desire to consume the drug. In specific embodiments, the reinforcing behavior is not included in addiction-related self-administration of the drug.

In certain embodiments, the medication is a replacement for the drug. In other embodiments, the medication decreases a craving of the patient for the drug. In still other embodiments, the medication causes an aversive effect in the patient in combination with the drug. In particular embodiments, the medication activates dopamine receptors, either directly or indirectly, by increasing levels of dopamine in the brain. In specific embodiments, the drug is selected from the group consisting of alcohol, amphetamines, cannabis, cocaine, hallucinogens, inhalants, opioids, phencyclidine, sedatives, hypnotics, and anxiolytics. For example, the drug is alcohol and the medication is naltrexone, or the drug is cocaine and the medication is a 3-phenyltropane, or the drug is alcohol and the medication is a 3-phenyltropane.

Another aspect of the invention provides a method of treating a patient for addiction to a drug other than nicotine. The method includes providing a medication for treating the addiction, identifying at least one reinforcing behavior, and instructing the patient to self-administer the medication and perform the reinforcing behavior concurrently between about three and about twenty times per day. In certain embodiments, the medication includes the drug in a formulation for decreasing the dependence of the patient on the drug.

The phrase “addiction to a drug,” as used herein, means the continued use of a specific psychoactive substance despite physical, psychological, or social harm. “Recovery” from addiction to a drug refers to the reduction or cessation of such harmful use of a psychoactive substance.

### DETAILED DESCRIPTION

The present invention provides methods for the treatment of addiction that include frequent or episodic dosing of medication coupled with a reinforcing behavior and/or stimulus. Such methods provide comprehensive addiction treatments that address both the pharmacological and behavioral aspects of a broad range of addictive disorders, thereby synergistically increasing the effectiveness of treatment over the use of medication alone. Performing a particular reinforcing behavior and/or experiencing a particular reinforcing stimulus associated with medicine delivery helps patients to become fully engaged in the process of overcoming addiction by encouraging patients mentally to focus on the existence of the addiction and the recovery process. The reinforcing behavior or stimulus also helps to increase the magnitude of the conditioned response that a patient develops to the dosing of medication, and to increase the efficiency of teaching the patient a new response to cravings associated with addiction. The new medication regimen associated with addiction therapy also imposes a schedule in the life of the patient, which facilitates initial recovery and helps the patient avoid relapse by providing an alternative to behaviors associated with the addiction. Further, dosing of medication in conjunction with a reinforcing behavior or stimulus often creates a short-term alternate dependency that aids in diminishing the original addiction. This short-term alternate dependency aids in the transition to community reinforcers and acts as a safety net in case of later risk of relapse.

The methods of the invention include administration of a medication having a pharmacological effect that helps a patient to overcome an addiction. Suitable medications for use in the methods of the invention include rapid onset psychoactive drugs, slow onset psychoactive drugs, and non-psychoactive medications. Rapid onset psychoactive drugs quickly induce an active response in a patient. For example, such medications help to reduce cravings for a drug to which a patient is addicted and/or provide relief from withdrawal symptoms. Slow onset psychoactive drugs do not induce an immediate effect in a patient and require a longer period of time before peak effect is achieved. For example, many anti-depressants, such as selective serotonin re-uptake inhibitors, take many weeks to achieve peak clinical activity. Non-psychoactive medications, which have rapid or slow onset, do not directly induce a psychoactive response. For example, such drugs block an abused drug from entering the brain or interfere with or enhance metabolism of an abused drug.

Medications that are particularly useful in the methods of the invention have a low potential for abuse in the final dosage form. “Low potential for abuse” means that a patient is unlikely to develop a pattern of recurrent substance use that interferes with the patient's ability to perform personally, e.g., fulfill family obligations, or socially, e.g., function properly at work. Especially useful medications are tolerated across a wide range of doses. As the methods of the invention often provide for frequent administration of medication, it is possible that a patient will exceed the recommended dose of medication. Tolerance across a wide range of doses means that no harm will be done to a patient if he or she exceeds the recommended dose by about 2- to 3-fold. Particularly suitable medications also have a low potential for overdose or are self-limiting, as is nicotine, meaning that a patient is unlikely to consume a sufficient quantity of the drug to cause acute physical harm. In some instances, the medication used for treating addiction is provided in combination with additional therapeutic agents, such as, for example, vitamins and/or other dietary supplements.

Some medications suitable for use in the methods of the invention are reinforcing to promote compliance. Such reinforcing medications themselves have some addictive properties, thus encouraging patients to take the medications. Non-limiting examples of such medications include methadone and LAAM for the treatment of heroin addiction, methylphenidate for the treatment of cocaine addiction, and gamma-hydroxybutyric acid for the treatment of alcoholism. Often, the medications are chosen to be only weakly reinforcing, such that patients easily transition off of the medications after treatment. Some suitable medications for use in the methods of the invention decrease a patient's desire to consume a drug to which the patient is addicted. For example, a patient's cravings are reduced by a medication that decreases the pleasurable effects associated with consuming the drug. Alternatively, drug consumption is discouraged by a medication that causes an aversive effect, such as nausea, upon drug consumption. For example, naltrexone is useful for helping patients to abstain from consuming alcohol or heroin or other opioids; disulfiram is useful for helping patients to avoid alcohol or cocaine; and acamprosate, Pueraria (Kudzu), bromocriptine, gamma-hydroxybutyrate (GHB), and serotonergic agents, such as ondansetron, ritanserin, and buspirone, are useful for assisting patients in abstaining from alcohol consumption. Further medications useful in the methods of the invention activate a dopaminergic pathway and thereby encourage the learning of new behavior patterns, as dopamine enhances learning and memory formation (Suzuki et al., J. Neurosci. 21:6492-6501 (2001); Thomas et al., J. Neurosci. 20:5581-5586 (2000)). Non-limiting examples include nicotine, cocaine, methylphenidate, amphetamine, caffeine, 3-phenyltropanes, bupropion, bromocriptine, and monoamine oxidase (MAO) inhibitors.

Some medications suitable for use in the methods of the invention are replacement or substitute drugs, i.e., drug alternatives that are, for example, less toxic, less psychoactive, less addictive, less likely to be abused, or formulated in a more controllable dosage form, but still provide some of the effects of a drug to which a patient is addicted. Non-limiting examples of such replacement drugs include methadone and buprenorphine for treating addiction to heroin or other opioids, and methylphenidate and phenyltropanes for treating cocaine addiction. In particular embodiments, the replacement drug is a chemical analog of the addictive drug. For example, analogs of cocaine, amphetamines, and opiates are useful in treating addictions to those drugs. Non-limiting examples of useful analogs include phenyltropanes for cocaine and buprenorphine for heroin.

Other suitable medications for use according to the methods described herein include a drug to which a patient is addicted, for example, nicotine in smoking cessation therapy. In at least some instances, the addictive drug is administered in a formulation designed to decrease the patient's dependence on the drug. For example, such a formulation includes a sufficiently small quantity of the drug to avoid reinforcing addictive behaviors. Another example is a formulation whose delivery route differs from addiction-related drug delivery routes, so that the drug enters the brain less rapidly than it does during addiction-related use. Further examples are formulations including additional components that hinder injection or other unintended uses of the drug, modify the effect of the drug, or decrease the ability of the drug to reinforce the addiction.

Non-limiting examples of medications suitable for use with methods of the invention include serotonin receptor antagonists, such as ondansetron, clozapine, ritanserin, ketanserin, meulergine, and tropisetron; serotonin receptor agonists, such as buspirone, gepirone, cisapride, ipsaperone, sumatriptin, and renzapride; serotonin re-uptake inhibitors, such as sertraline, venlafaxine, fluoxetine, paroxetine, citalopram, and fluvoxamine; norepinephrine re-uptake inhibitors, such as amitryptyline, clomipramine, doxepin, imipramine, trimipramine, amoxapine, desipramine, maprotiline, nortryptyline, and protryptyline; atypical antidepressants, such as bupropion, nafazadone, and trazadone; monoamine oxidase inhibitors, such as phenelzine, tranylcypromine, and selegiline; dopaminergic agents, such as tiapride, methylphenidate, mazindol, pemoline, nomefensin, and bromocriptine; GABA agents, such as baclofen, GHB, and gamma-vinyl-GABA; NMDA receptor agents, such as dextromethorphan, phencyclidine, memantine, and acamprosate; opioid antagonists, such as naltrexone and nalmefene; and agents that interfere with the metabolism of alcohol, such as disulfiram and calcium carbimide.

Non-limiting examples of dosages and treatment schedules for use of particular medications according to certain embodiments of the invention are set forth below. Such medications are useful in treating alcoholism and addiction to other drugs, such as, for example, opiates and stimulants, such as cocaine and methamphetamine. The particular dosages, routes of administration, proposed mechanisms of action, salt forms, etc. described for the medications below are exemplary, and not limiting of the invention.

Those of skill in the art will appreciate that the dosage and treatment schedule for a given medication will vary, for example, based on method of delivery and patient characteristics, and are to be determined by a physician (see, e.g., Harrison's Principles of Internal Medicine, 15^{th} Ed., Braunwald et al., eds., McGraw-Hill Professional (2001)). The exemplary dose ranges listed below are intended for a typical adult of average weight, i.e., between about 55 kg and about 90 kg, typically about 70 kg. In the art, doses are usually selected to attain a particular target concentration of medication in a patient. The dosing interval and amount of medication per dose are selected so that medication levels never exceed a maximum safe concentration, but a convenient dosing schedule is still possible.

In some embodiments of the methods described herein, the chosen medication dose is less than the amount required to fully satisfy a patient's cravings for an extended period of time and/or substantially less than the maximum safe dosage, such that the medication must be dosed frequently enough to engage the patient in the reinforcing behavioral component of medication delivery and treatment. In such embodiments, dosages are purposefully reduced so that the reinforcing behavior associated with medication delivery, described in more detail below, must be more frequently repeated. Such behavioral engagement helps the patient to focus on the existence of the addiction and the goal of recovery, and thus to take ownership of the treatment plan and the recovery process.

In certain embodiments, medications are dosed between about 3 times and about 20 times per day, for example, between about 4 times and about 15 times per day, between about 4 times and about 10 times per day, or between about 6 times and about 9 times per day. In some instances, treatment is continued for about 6 weeks to about 52 weeks, for example, for about 12 weeks to about 26 weeks, or from about 12 weeks to about 16 weeks. In some embodiments, treatment is continued on an episodic basis as needed, for example, throughout the life of the patient, in response to craving and/or to prevent relapse.

One example of a medication suitable for use in treating addiction according to the methods described herein is naltrexone. Naltrexone is an opiate receptor antagonist that is available as the hydrochloride salt. Naltrexone is approved for treating alcoholism and also is useful, for example, in treating opiate addiction. Suitable daily doses range, for example, between about 20 mg and about 80 mg. In certain embodiments, a daily dose of about 50 mg is used. Other opiate receptor antagonists, such as, for example, naloxone and nalmefene, are also useful in addiction therapy.

Another suitable medication is bupropion, which is useful in treating addiction to drugs including, for example, nicotine. Bupropion is an aminoketone that is available as the hydrochloride salt or as a sustained release formulation. Bupropion is suitable for oral, transmucosal, or transdermal administration. In certain embodiments, a typical daily dose is about 300 mg, for example, between about 200 mg and about 400 mg, but not more than about 450 mg.

Still another example is bromocriptine, a dopamine receptor agonist that is useful, for example, in treating alcoholism or addiction to a stimulant such as cocaine or methamphetamine. Bromocriptine is suitable for oral delivery, and in some instances is administered in an amount between about 1 mg/day and about 20 mg/day, for example, between about 3 mg/day and about 17 mg/day, between about 5 mg/day and about 10 mg/day, or about 7.5 mg/day.

Acamprosate is another medication that is useful for treating addictions including, but not limited to, alcoholism. In some embodiments, acamprosate is administered orally, with a daily dose between about 500 mg and about 8 g, for example, between about 1 g and about 5 g, or between about 1.3 g and about 2 g.

Still another non-limiting example of a medication useful in the methods described herein is disulfiram, which is useful, for example, in treating alcoholism or addiction to a stimulant, such as cocaine or methamphetamine. Disulfiram is suitable for oral administration. In some embodiments, disulfiram is delivered in an amount between about 50 mg/day and about 2000 mg/day, for example, between about 100 mg/day and about 1000 mg/day, or between about 250 mg/day and about 500 mg/day.

Amphetamine, a central nervous system stimulant, is another medication suitable for use in the methods described herein. Amphetamine is useful, for example, in treating addiction to a stimulant, such as cocaine or methamphetamine. Various salt forms of amphetamine are suitable for use according to the methods described herein, including, but not limited to, amphetamine sulfate, phosphate, or aspartate. Dextroamphetamine, in both the free base and the salt form, is useful in the methods described herein. Amphetamine is suitable for oral, transdermal, or transmucosal administration. In certain embodiments, amphetamine is administered in a daily dose ranging from about 2 mg to about 100 mg, for example, from about 5 mg to about 75 mg, from about 30 mg to about 100 mg, or from about 30 mg to about 36 mg.

Another useful medication is methylphenidate, a mild central nervous system stimulant that is available as the hydrochloride salt or in a sustained release formulation and is suitable for oral delivery. In certain embodiments, methylphenidate is administered in the form of a tablet. In other embodiments, methylphenidate is administered as a formulation that is sprinkled on food. Methylphenidate is suitable, for example, for treating addiction to a stimulant, such as cocaine or methamphetamine. Also useful in treating addiction are methylphenidate analogs having comparable central nervous system stimulant activity to methylphenidate, including those analogs that cause a slower onset of action than methylphenidate. Methylphenidate has an elimination half-life of about 2 to 3 hours, a time to peak plasma concentrations of about 1 to 3 hours, and a 3 to 4 hour duration of behavioral effect. In certain embodiments, methylphenidate is administered in an amount between about 2 mg/day and about 100 mg/day, for example, between about 5 mg/day and about 60 mg/day, between about 10 mg/day and about 50 mg/day, or between about 20 mg/day and about 40 mg/day.

Yet another useful medication is ondansetron, a selective 5-HT3 receptor antagonist that is available either as the base or in a salt form, such as the hydrochloride dihydrate. In certain embodiments, ondansetron is used to treat alcoholism, as it reduces the cravings of early onset alcoholics for alcohol. In particular embodiments, ondansetron is administered orally at a daily dose ranging between about 0.1 mg and about 50 mg, for example, between about 0.2 mg and about 24 mg, between about 0.5 mg and about 3 mg, between about 1 mg and about 5 mg, or between about 2 mg and about 10 mg.

Another useful medication, caffeine, is a competitive adenosine receptor antagonist that is suitable for oral or transdermal delivery. In certain embodiments, caffeine is administered at a daily dose ranging between about 10 mg and about 1500 mg, or between about 20 mg and about 2000 mg, for example, between about 50 mg and about 750 mg, between about 75 mg and about 500 mg, or between about 100 mg and about 1000 mg.

Another suitable medication for use in accordance with the methods described herein is cocaine, which is useful in various salt forms, such as the hydrochloride, nitrate, or sulfate. Cocaine is suitable for oral or transdermal administration. In certain embodiments, cocaine is administered in a daily dose ranging from about 20 mg to about 5000 mg, for example, from about 40 mg to about 2000 mg, from about 100 mg to about 1000 mg, or from about 200 mg to about 600 mg. Further useful medications include synthetic cocaine analogs that have comparable central nervous system stimulant activity to cocaine, including those analogs that cause a slower onset of action than cocaine. Cocaine and its analogs are useful, for example, in treating addiction to a stimulant such as cocaine or methamphetamine. A non-limiting example of a useful class of cocaine analogs is the 3-phenyltropanes, which have a high affinity for the neurotransmitter reuptake inhibitors. RTI-336 is a 3-phenyltropane that has high affinity and selectivity for the dopamine transporter. RTI-336 is orally available, and studies have demonstrated that RTI-336 inhibits cocaine self-administration in rats. Rats pre-treated with RTI-336 (53.8 mg/kg, oral) decreased their willingness to press a lever for a cocaine infusion from 150±20 lever presses to 36±14 lever presses (unpublished observations, Dr. Susan Schenck, Victoria University of Wellington).

Yet another suitable medication, nicotine, is a tertiary amine with broad pharmacological activity. Nicotine is suitable for use in smoking cessation therapy, and also in treating other drug addictions. In certain embodiments, nicotine is used in combination with another medication for treating an addiction other than nicotine dependence. Nicotine is used to control dosing of the second medication. Patients are motivated to take a dose of the second medication because the accompanying nicotine alleviates nicotine withdrawal, and thus also promotes smoking cessation. The nicotine also prevents patients from exceeding the recommended dose of the second medication, because too much nicotine causes nausea and light-headedness. Various salt forms of nicotine are suitable for use according to the methods described herein, including, but not limited to, nicotine salicylate or bitartrate. Nicotine is suitable for oral, transdermal, buccal, or intranasal administration, with daily dosages ranging, for example, from about 2 mg to about 200 mg, from about 5 mg to about 150 mg, from about 10 mg to about 100 mg, or from about 15 mg to about 75 mg.

The efficacy of a medication often is influenced by the mode of its administration. For example, studies with respect to the delivery of placebos have demonstrated that the color of the dosage form, as well as the frequency and route of administration, can influence the magnitude of the placebo effect (de Craen et al., BMJ 313:1624-1626 (1996); de Craen et al., Br. J. Clin. Pharmacol. 48:853-860 (1999); Kaptchuk et al., J. Clin. Epidemiol. 53:786-792 (2000)). Dosing of a medication in accordance with the methods described herein promotes the treatment of addiction with increased effectiveness beyond that provided by the direct pharmacological effect of the medication alone.

In some embodiments, medication dosing is scheduled and frequent, for example from about three to about twenty times per day. In certain embodiments, dosing occurs at least about four times per day, for example, between about five and about ten times per day. In particular embodiments, dosing occurs at least about eight times per day, for instance, from about ten to about twelve times per day. Often the number of scheduled daily doses is tapered gradually over the course of addiction treatment. For example, the scheduled frequency of delivery is reduced by about 1 to about 2 units per day over a treatment period of about 12 weeks, or the patient is instructed to skip those doses taken at particular periods of craving (e.g., after a meal for smoking or before dinner for alcohol consumption).

Scheduled, frequent dosing of medication during treatment is useful even when addiction-related drug use by a patient is less frequent or sporadic, for example, occurring only once per day or at differing intervals in response to cravings or situational cues. Adopting an organized, structured lifestyle often helps a recovering addict to overcome addiction and avoid relapse (see, e.g., Therapy Manuals for Drug Addiction: An Individual Drug Counseling Approach to Treat Cocaine Addiction, Chapter 8, National Institute on Drug Abuse (1999)). The addict often needs to find alternative behaviors to fill the time previously spent performing activities associated with procuring and administering the addictive drug. Frequent dosing of medication provides a useful way to impose a new routine in the life of a recovering addict, and to reinforce the emphasis placed on structure and time management by some behavioral approaches to addiction therapy. Further, frequent dosing sometimes is used as part of a behavioral treatment program that emphasizes adherence to treatment, such as contingency management.

In some alternative embodiments, dosing is episodic. For example, medication is delivered as needed during or in anticipation of cravings, or when the effects of a previous dose of medication wear off. Sometimes, a medication that causes unpleasant effects in combination with a drug to which a patient is addicted is administered in anticipation of or in conjunction with drug use (see, e.g., Sinclair, Alcohol Alcohol. 36:2-10 (2001)). In some instances, medication is administered episodically as needed over the life of a patient whenever relapse threatens. In certain embodiments, episodic dosing is used following one or more periods of more frequent or scheduled dosing. After a period of frequent dosing, episodic dosing is continued as needed, for example, throughout the life of the patient, in response to craving or whenever relapse threatens.

Episodic dosing is useful for reinforcing the skills training and coping strategies that are important in some behavioral approaches for treating addiction. Episodic dosing is particularly useful, for example, when the goal is to increase a patient's cognitive control over his or her addiction. In some instances, episodic dosing is part of a behavioral treatment program that emphasizes coping skills. Taking medication in response to craving is one skill that is taught during therapy. Episodic dosing also is especially useful, for example, late in therapy or after the cessation of intensive therapy when cravings only occur intermittently. Further, episodic dosing is sometimes used for a medication that has a rapid effect on craving or response to the addictive drug. In contrast, scheduled frequent dosing is particularly useful for medications requiring a longer time to reach peak effect, such that a patient receives less immediate feedback from the medication itself.

Delivery of medication according to the methods of the invention is coupled with a particular reinforcing behavior. The reinforcing behavior is an action that is repeatedly performed concurrently with medication delivery, and thus becomes associated with medication delivery and addiction therapy. In certain embodiments, the reinforcing behavior is an action that is part of self-administering the medication, for example, chewing a medicinal lozenge or rubbing on a topical formulation. In other embodiments, the reinforcing behavior is a separate and/or unrelated action. The reinforcing behavior often becomes ritualized as a part of addiction treatment and often is associated with a sensory stimulus unrelated to-the pharmacological effect of the medication. In at least some instances, the medication is formulated such that delivery of the medication and performance of the reinforcing behavior provides a sensory stimulus, such as, for example, a taste provided by eating a medicinal lozenge, a heat or cold sensation caused by rubbing on a topical medicine formulation, or a tingling sensation provided by placing an effervescent medication tablet in the mouth. Performing the reinforcing behavior and experiencing the sensory stimulus in conjunction with medication delivery provide important behavioral components in addiction therapy. The reinforcing behavior and stimulus encourage the patient mentally to focus on recovery by serving as recurrent reminders that the patient has an addiction that he or she is working to overcome. In addition, if the reinforcing behavior becomes ritualized, medication delivery is controlled by portions of the brain that govern automatic behaviors rather than conscious cognitive behaviors. Thus, the ritualized behavior competes more effectively with the behaviors associated with drug taking and addiction. A ritualized behavior is one which initially requires conscious cognitive attention to perform but becomes automatic and/or habitual when repeated many times.

The reinforcing behavior and stimulus coupled with medication delivery during therapy help to create an alternate dependency that assists a patient in overcoming an original addiction. When the addiction that the patient wishes to overcome includes a behavioral aspect, the reinforcing behavior associated with treatment provides a replacement behavior that allows the patient to abandon the behavior associated with addiction. For example, delivering nicotine orally in liquid form through a straw-like delivery device becomes a substitute for the hand-to-mouth behaviors associated with cigarette smoking. Some addictive disorders, such as addiction to cocaine, do not include a substantial behavioral component associated with drug delivery that reinforces the addiction. That is, although there is inherently a behavior associated with addiction-related self-administration, the behavior itself is not reinforcing without the pharmacological effect of the drug. However, environmental cues are still very important in inducing craving for the addictive drug, and such addictions are associated with serious behavioral disruptions. Therefore, a positive reinforcing behavioral component should be included in an addiction treatment program of the invention. The goal of such treatment programs is to replace unhealthy addiction-related behavioral patterns with reinforcing behaviors that are associated with medication delivery during treatment and to encourage the patient to engage in treatment and focus on recovery.

Sometimes, the reinforcing behavior coupled with medication delivery during therapy has some similarities to activities associated with a patient's addiction, or is an excessive exacerbation of such activities. Behavioral similarities between the activities ease a patient's transition from practicing the addiction to engaging in therapy. However, the reinforcing behavior should not be so similar as to reinforce behaviors associated with addiction. Thus, the reinforcing behavior should not be identical to a behavior associated with addiction, and sometimes is completely different from or unrelated to behaviors associated with addiction. For example, in one embodiment, the chosen method for administration of medication during treatment is different from a method of addiction-related self-administration, so that behavioral distinctions between addiction therapy and the underlying addiction are reinforced. Addiction-related self-administration refers to a method used by a patient to deliver a drug to which he or she is addicted. Examples of addiction-related self-administration include, but are not limited to, smoking cigarettes to deliver nicotine, insufflating a powder through a tube or inhaling a sublimated form through a pipe to deliver cocaine, and insufflating a powder or injecting a solution with a syringe to deliver heroin. The frequency of addiction-related self-administration varies by drug, patient, and route of administration. For example, some heavy smokers smoke about 80 cigarettes per day, corresponding to a relatively constant and continual inhalation of nicotine (one cigarette every 10 minutes, one puff per minute). Heroin addicts typically inject heroin about every 3 to 4 hours every day. Cocaine addicts typically binge on cocaine, using the drug approximately every 15 minutes for about 8 to about 24 hours approximately once or twice per week. Alcoholics typically either drink constantly throughout the day or binge approximately 4 to 5 times per week, ingesting up to about 20 drinks in a period of approximately 4 to 5 hours.

Sometimes, self-administration of medication during treatment is associated with a particular reinforcing sensory stimulus that, similarly to the reinforcing behavior, reminds the patient of the addiction and engages the patient in the recovery process. In at least some instances, the sensory stimulus becomes a conditioned reinforcer that enhances the efficacy of a medication after repeatedly being associated with medication dosing. The sensory stimulus is a taste, smell, sight, sound, or tactile sensation that the patient experiences concurrently with medication delivery during treatment. Often the sensory stimulus is not associated with addiction-related self-administration of the drug to which the patient is addicted. Thus, the stimulus provides a reinforcing sensory distinction between addiction and treatment that helps the patient mentally to focus on recovery. Often, the stimulus also is unrelated to the pharmacological effect of the treatment medication. For example, delivery of medication in a tea provides multiple stimuli such as the taste and aroma of the tea, the warmth of the tea cup, and the sound of the whistle on the tea kettle. Such stimuli are not associated with addiction-related drug administration or the effect of therapy medication, and provide a particular repeated set of sensory cues that the patient comes to associate with treatment and depend upon throughout the process of recovery.

Nicotine replacement therapy techniques provide examples of drug delivery mechanisms having reinforcing behavioral and stimulus components. Nicotine is delivered as a patient performs a particular behavior, for example sucking a liquid suspension of nicotine granulate through a straw-like nicotine delivery device. Such a technique combines nicotine delivery with tactile and oral stimuli and a reinforcing behavior. While providing a useful vehicle for nicotine replacement therapy, oral ingestion through a straw-like device is useful for delivering drugs for the treatment of any type of addiction, alone or in combination. In some instances, a medication for treating an addiction other than nicotine dependence is delivered through a straw-like device along with nicotine. The nicotine serves to control the dosing of the other medication, as described above. In at least some such embodiments, the patient is a smoker who is also addicted to a drug other than nicotine. Thus, the combination therapy including nicotine serves to promote smoking cessation as well as treating the other addiction. As a non-limiting example, a straw that delivers naltrexone and nicotine is used to treat patients who are both alcoholics and smokers. The combination of pharmacological and behavioral treatments provided by frequent dosing of nicotine and naltrexone via a straw delivery device synergistically helps patients to stop drinking and smoking. Beyond simply providing useful doses of nicotine and naltrexone, frequent dosing with the nicotine-naltrexone straw causes patients to focus on recovery, reinforces new responses to alcohol and tobacco cravings, and provides patients with a tool to address episodic cravings.

Other routes for delivering medication according to methods of the invention include, but are not limited to, oral vehicles such as a sublingual tablet, a mouth rinse or gargle, a mouth spray, a toothpaste, a toothpick, a chewing gum, a composition licked from a stamp or other support material, a solid dosage form that effervesces in the mouth (e.g., Pop Rocks®), a candy, such as a chocolate or caramel chew, and crackers or other food. Each of these methods for medication delivery provides an oral stimulus unrelated to the medication and requires particular actions of the hands and mouth to accomplish medication delivery. At least some of these medication delivery vehicles also provide a particular smell and/or a sound associated with, for example, opening the package of the dosage formulation or chewing a food that includes the medication. Further examples of drug delivery vehicles useful for practicing the methods the invention include beverage additives such as a tea, a coffee creamer, or an effervescent tablet. The aroma, taste, and oral sensation of the beverage provide sensory stimuli, and preparing and drinking the beverage are reinforcing behaviors associated with medication delivery. Other suitable medication delivery vehicles include eye drops or nasal spray, such that administration is associated with a dispensing behavior and a sensation in the eyes or nose.

Transdermal routes for administration are also useful in the methods of the invention, for example administration of medication in a body oil, lotion, gel, mousse, hairspray, aftershave, nail polish, lip balm, or perfume. Further vehicles for transdermal administration include an aerosol or pump spray; a comb or brush that releases medication to the scalp during use; a device worn by a patient, such as a watch band, ring, bracelet, or patch, that releases medication when pressed or tapped by the patient; and a device for manipulation by a patient, such as a “worry stone,” “worry beads,” or “stress ball,” that releases medication when squeezed or rubbed. Each of these vehicles requires a particular behavior for administration and is associated with tactile and sometimes also olfactory sensations. In some embodiments, the behavioral aspect of transdermal administration is enhanced, for example by encouraging a patient to trace a meditative pattern or words with a composition that is absorbed transdermally, or to rub a “scratch card” that is coated with a transdermally absorbed composition, possibly revealing a message, or prize. Alternatively, the stimulus associated with transdermal administration is enhanced, for example, by administering the medication along with an agent that generates heat or feels cold, indicating to the patient that the medication is working.

Repetition at regular intervals of a reinforcing behavior or stimulus associated with medication delivery during treatment serves as a behavioral tool, helping the patient mentally to focus on addiction and recovery. Studies have shown that the pharmacological effect of a drug can become a conditioned response evoked by a particular cue that is itself pharmacologically neutral, but has been reliably associated with drug administration (Ader, “The Role of Conditioning in Pharmacotherapy” in The Placebo Effect: An Interdisciplinary Exploration, Harrington, ed., Harvard Univ. Press, 138-165 (1997)). The repeated association of a particular behavior or stimulus with medication delivery according to the methods described herein serves to increase the magnitude of the conditioned response created by administering a medication, thereby increasing the efficacy of the medication itself. Sometimes, the reinforcing behavior and stimulus create a competing dependency that helps to extinguish the original addiction. The usefulness of repeated behaviors in treating addiction is supported by smoking cessation meta-analyses, which indicate that the nicotine patch is a less effective treatment for nicotine addiction than alternative treatments that have a behavioral component, e.g., nicotine gum, inhaler, and nasal spray (see, Nicotine Replacement Therapies in Smoking Cessation: A Review of Evidence and Policy Issues, Canadian Council on Tobacco Control). This finding contradicts the traditional view that frequent dosing is disfavored, and that formulations requiring less frequent dosing, including depot formulations such as the nicotine patch, will be more effective due to increased patient compliance. However, this unexpected result is consistent with the methods of the invention, which provide for the treatment of addiction by frequent or episodic dosing of medication coupled with a reinforcing behavior or stimulus.

The addiction treatment methods of the invention often are carried out in conjunction with patient counseling to encourage a full recovery of mental and physical health. Counseling methods include, but are not limited to, cognitive behavioral therapy, motivation to change, contingency management, individual psychotherapy, group therapy sessions, in-patient programs, out-patient therapy, intensive out-patient therapy, extinction of conditioned craving, coping skills therapy, network therapy, aversion therapy, community reinforcement, and “twelve-step” programs. Sometimes, the methods of the invention are practiced simultaneously with more traditional pharmacological methods for addiction therapy. For example, a nicotine replacement method having a behavioral component, e.g., nicotine straw, is used in combination with daily doses of a pharmacological agent such as bupropion to provide an optimized treatment regimen for smoking cessation. Another non-limiting example is the use of short-acting anti-craving medication in association with daily doses of naltrexone for alcoholism.

The methods of the invention are useful for treating essentially any type of drug addiction. For example, the methods are useful for treating addictions to drugs such as alcohol, amphetamines, cannabis, cocaine, hallucinogens, inhalants, opioids, phencyclidine, sedatives, hypnotics, and anxiolytics, which are subject to abuse, promote dependence, and cause intoxication. The methods of the invention also are useful for treating dependence on drugs such as nicotine, which are not subject to abuse and do not cause intoxication. Further, the methods of the invention are useful for treating non-drug addictive disorders, such as gambling and obesity, for which pharmacotherapy is indicated. Additional applications include the treatment of any chronic disease where medication compliance is difficult, such as AIDS, tuberculosis, hypertension, asthma, diabetes, or high cholesterol. The ritualization of medication delivery through the methods of the invention helps to make self-administration of medication automatic or habitual, thereby increasing patient compliance.

The following non-limiting examples further illustrate certain embodiments of the invention:

### EXAMPLE 1

Nicotine-Naltrexone Straw

1. Device

Nicotine and naltrexone are administered orally using a straw-like oral delivery device as described in detail in co-pending and co-assigned U.S. patent application Ser. No. 10/045,235 and the continuation-in-part application thereof entitled “Device and Method for Treating Smoking and Alcoholism” filed on even date herewith. The delivery device provides medications for treating addiction, while also providing oral and tactile stimulation. Briefly, the device includes a tubular chamber in the form of a plastic drinking straw. The tubular chamber contains nicotine and naltrexone. The nicotine and naltrexone are in the form of coated sugar spheres that include nicotine bitartrate or naltrexone hydrochloride. Each device contains 8 mg nicotine and 10 mg naltrexone. The medications are contained within the straw by a removable cap at one end and a filter at the other end of the straw. The user removes the cap, places the end of the straw having the filter in a glass of apple juice, and applies oral suction to the other end of the straw. Upon application of oral suction, the juice, nicotine, and naltrexone are delivered into the user's mouth.

2. Addiction Therapy

Patients diagnosed as alcoholics and smokers are instructed to use the device as needed throughout the day to administer a dose of nicotine and naltrexone in response to cravings for either tobacco or alcohol. Patients are instructed not to exceed one dose every 1.5 hours or 10 doses per day. Patients are monitored for progress toward cessation of smoking and/or drinking alcohol, i.e. for changes in smoking and/or alcohol consumption levels.

### EXAMPLE 2

Carbonated Naltrexone Product for Alcoholism

1. Candy Production

A sugar melt is produced using a mixture of sucrose, lactose, and corn syrup in a weight ratio of 52:27:21. The mixture is dissolved in water and evaporated at a temperature of 320° F. to yield a moisture content of about 3%. The melt is placed in a pre-heated pressure vessel, and naltrexone hydrochloride is added to generate a final preparation having 10 mg naltrexone per gram of final product. The vessel is placed in a controlled temperature bath and pressurized with CO₂ at 750 psig for 5 minutes with vigorous mixing. The vessel is cooled to solidify the sugar melt under pressure. The pressure is released rapidly, thus fracturing the solidified carbonated product into multiple pieces. The product is sieved to generate pieces of roughly uniform size. One serving or dose contains approximately 10 mg of naltrexone in 1 g of candy product. The gasified naltrexone product is packaged in a protective wrapper to maintain low water content.

2. Addiction Therapy

Patients diagnosed with alcohol dependence are treated using the carbonated naltrexone product. Qualified subjects are free from opiates for at least 7 days prior to use of the naltrexone product. Patients are instructed to place the carbonated naltrexone product in their mouth when they experience craving for alcohol. When placed in the mouth, the gasified candy produces a distinct “popping” sensation, while simultaneously delivering naltrexone. Dosing at least 4 times per day, but not more than 10 times per day, is recommended. Dosing is continued for at least 12 weeks, and is continued for up to 26 weeks if judged necessary by the attending physician. Following this treatment period, subjects are directed to continue using the naltrexone product on an ad lib basis as needed to deal with spontaneous or induced cravings for alcohol.

### EXAMPLE 3

3-Phenyltropane for Cocaine Addiction

1. Dose Ranging Studies

The 3-phenyltropane compound RTI-336 is formulated in a tablet at a dose suitable for use in humans. The appropriate dose is selected in a series of dose escalating clinical trials in humans. Multiple dosages are tested, including doses between 0.5 mg/kg and 25 mg/kg. The trials address first the safety and tolerability of the product, as judged by vital signs and clinical chemistries, and then its efficacy, as measured by reduction in cocaine use.

2. Addiction Therapy

Patients diagnosed with cocaine dependence are treated with RTI-336, using dosage levels determined as described in part A above. RTI-336 is provided as a tablet together with a 20 ml vial of flavored solution. Several flavors of solution are available, such as vanilla, cardamom, and eucalyptus, allowing each patient to choose an appealing flavor/aroma that is not commonly encountered in his everyday activities. Patients are instructed to swallow the RTI-336 tablet using the flavored solution when they experience craving for cocaine. Dosing at least 4 times per day, but not more than 10 times per day, is recommended. Dosing is continued for at least 12 weeks, and is continued up to 26 weeks if deemed necessary by the attending physician. Following this treatment period, it is recommended that patients administer the solution (with or without the RTI-336 tablet) on an ad lib basis to deal with cravings for cocaine.

Equivalents

While the foregoing invention has been described in some detail for purposes of clarity and understanding, it will be appreciated by one skilled in the art from a reading of this disclosure that various changes in form and detail can be made without departing from the scope of the invention.

What is claimed is:

1. A method of treating a patient for addiction to a drug, the method comprising:
(a) providing a medication other than the drug for treating the addiction;
(b) identifying at least one reinforcing behavior; and
(c) instructing the patient to self-administer the medication and perform the reinforcing behavior concurrently between about three and about twenty times per day.

2. The method of claim 1, wherein self-administration of the medication provides a reinforcing sensory stimulus.

3. The method of claim 2, wherein the sensory stimulus is unrelated to addiction-related self-administration of the drug.

4. The method of claim 2, wherein the sensory stimulus is unrelated to the pharmacological effect of the medication.

5. The method of claim 2, wherein the reinforcing behavior is included in self-administration of the medication.

6. The method of claim 1, wherein self-administration of the medication is more frequent than addiction-related self-administration of the drug.

7. The method of claim 1, wherein self-administration of the medication is performed at least five times per day.

8. The method of claim 7, wherein self-administration of the medication is performed at least eight times per day.

9. The method of claim 1, wherein the patient is instructed to self-administer the medication and perform the reinforcing behavior upon experiencing craving for the drug.

10. The method of claim 1, wherein the reinforcing behavior is not included in addiction-related self-administration of the drug.

11. The method of claim 1, wherein the medication is a replacement for the drug.

12. The method of claim 1, wherein the medication decreases a craving of the patient for the drug.

13. The method of claim 1, wherein the medication causes an aversive effect in the patient in combination with the drug.

14. The method of claim 1, wherein the medication activates dopamine receptors.

15. The method of claim 1, wherein the drug is selected from the group consisting of alcohol, amphetamines, cannabis, cocaine, hallucinogens, inhalants, opioids, phencyclidine, sedatives, hypnotics, and anxiolytics.

16. The method of claim 15, wherein the drug is alcohol and the medication is naltrexone.

17. The method of claim 15, wherein the drug is cocaine and the medication is a 3-phenyltropane.

18. The method of claim 15, wherein the drug is alcohol and the medication is a 3-phenyltropane.

19. A method of treating a patient for addiction to a drug, the method comprising:
(a) providing a medication for treating the addiction;
(b) identifying at least one reinforcing behavior; and
(c) instructing the patient to self-administer the medication and perform the reinforcing behavior concurrently between about three and about twenty times per day,
wherein the drug is not nicotine.

20. The method of claim 19, wherein the medication is the drug in a formulation for decreasing the dependence of the patient on the drug.
